(19) United States
(12) Patent Application Publication
(10) Pub. No.: US 2018/0368905 A1
(43) Pub. Date: Dec. 27, 2018

(54) SALINE FIELD ELECTROSURGICAL SYSTEM

(71) Applicant: Scott T. Latterell, Minneapolis, MN (US)

(72) Inventor: Scott T. Latterell, Minneapolis, MN (US)

(21) Appl. No.: 15/780,062

(22) PCT Filed: Nov. 30, 2016
(86) PCT No.: PCT/US2016/064231

§ 371 Date: May 30, 2018

Related U.S. Application Data
(60) Provisional application No. 62/260,941, filed on Nov. 30, 2015.

(51) Int. Cl. A61B 18/12; A61B 18/04; A61B 18/14

(52) U.S. Cl. CPC A61B 18/1206; A61B 18/1233; A61B 2018/00601; A61B 18/14; A61B 18/042

(57) ABSTRACT

An electrosurgical generator for supplying radio frequency (RF) power to an electrosurgical instrument that has been introduced to the surgical site for cutting or vaporizing tissue immersed in a saline medium is disclosed.

This application is being filed as a PCT International Patent application on Nov. 30, 2016, in the name of Scott T. Latterell, a U.S. Citizen, applicant and inventor for the designation of all countries and claims priority to U.S. Provisional Patent Application No. 62/260,941, filed Nov. 30, 2015, the contents of which are herein incorporated by reference in its entirety.

### FIELD OF THE INVENTION

The present invention relates to electrosurgical generators for supplying radio frequency (RF) power to an electrosurgical instrument.

### BACKGROUND

Electrosurgery is a well-established technology for modification of soft tissues which relies on a radio frequency (RF) energy source with an output between 100 kHz and 1 MHz. However, a need exists for improved systems and methods for electrosurgery.

### SUMMARY OF THE INVENTION

The present application is directed, in part, to an electrosurgical generator for supplying radio frequency (RF) power to an electrosurgical instrument that has been introduced to the surgical site for cutting or vaporizing tissue immersed in a saline medium, the generator comprising: an internal RF delivery stage able to deliver more than 55 Joules of energy to the electrosurgical instrument within 110 ms; and an internal storage capacity associated with RF waveform supply of less 5 Joules.

In certain implementations the RF stage is able to deliver up to 110 Joules of energy within 145 ms

In certain implementations the RF stage is able to deliver up to 110 J of energy within 110 ms.

In certain implementations the RF stage is able to deliver up to 230 J of energy within 320 ms.

In certain implementations an RF waveform synthesis stage including at least 1 pair of RF switching transistors is incorporated.

In certain implementations, an RF synthesis stage including at least 2 pairs of RF switching transistors is incorporated.

In certain implementations the transistors are configured as an H bridge circuit comprised of 2 half bridge pairs of transistors.

In certain implementations a maximum peak RF voltage is less than 500V and a maximum root mean square voltage is less than 360 Vrms.

In certain implementations the maximum output current is in excess of 3A root mean square with a RF current measurement sensor coupled to a control circuit able to disable the unipolar (dc) supply to the RF stage within ½ of the RF cycle upon detection of an electrosurgical instrument current in excess of an allowable limit.

In certain implementations an RF current measurement sensor is coupled to a control circuit able to within ½ of the RF cycle, alter the switching pattern of the RF transistors such that the voltage difference between the centre point nodes of the 2 half bridge pairs remains substantially zero, but the impedance between centre point nodes via 2 of the 4 switching transistors remains less than 1 Ohm.

In certain implementations a means of computing the energy delivered to the electrode over a time interval is included, where the energy delivery rate is dropped to less than 300 W outside the specified power surge intervals.

In certain implementations incorporating a means of computing the energy delivered to the electrode over a time interval is incorporated, where the energy delivery rate is dropped to less than 160 W outside the specified power surge intervals.

In certain implementations a generator according with a time constrained power surge interval incorporates a means of computing the impedance between the electrode poles, where RF delivery is stopped upon detection of an unacceptable impedance indicative of an absent or incomplete vapor gap or plasma within the power surge interval or optionally an impedance settling delay thereafter; with an impedance settling delay of up to 1 second; and with an unacceptable impedance being one of less than 300 Ohms and preferably less than 600 Ohms.

In certain implementations a means of computing the impedance between the poles of the electrode where upon initial activation of RF delivery is incorporated, a during a diagnostic interval preceding commencement of RF treatment; an RF voltage of less than 180 Vrms is applied, with RF treatment commencing only if the measured impedance falls within an acceptable range.

In certain implementations the minimum acceptable impedance during the diagnostic time interval has a value between 10 and 180 Ohms.

In certain implementations the minimum acceptable impedance during the diagnostic time interval has a value between 20 and 180 Ohms.

In certain implementations the minimum acceptable impedance during the diagnostic time interval has a value between 100 and 180 Ohms.

In certain implementations the maximum acceptable impedance during the diagnostic time interval has a value between 20 and 400 Ohms.

In certain implementations the maximum acceptable impedance during the diagnostic time interval has a value between 20 and 60 Ohms.

In certain implementations a generator alternating between a first RF plasma delivery mode and a second RF non-plasma delivery mode; with the waveform voltage amplitude during the RF plasma delivery mode being greater than 220 Vrms; and the voltage during RF non-plasma delivery mode being less than 180 Vrms; wherein the generator remains in RF plasma delivery mode until an RF plasma mode impedance limit is measured to have been exceeded whereupon it switches to the RF non-plasma mode; and the generator remains in RF non-plasma mode until the impedance falls below a RF non-plasma mode impedance limit (indicative of plasma vapor gap collapse), or until a maximum non-plasma mode interval has elapsed.

In certain implementations a generator wherein the RF plasma mode impedance limit is greater than 750 Ohms is provided.

In certain implementations the RF plasma mode impedance limit is greater than 900 Ohms.

In certain implementations the RF plasma mode impedance limit is adjustable between 400 and 1600 Ohms by a sensitivity user adjustment.

In certain implementations the RF non-plasma mode impedance limit is less than 400 Ohms.

In certain implementations the RF non-plasma mode impedance limit is less than 120 Ohms.

In certain implementations the RF non-plasma mode impedance limit is adjustable between 40 and 600 Ohms via a sensitivity user adjustment.

In certain implementations the maximum non-plasma mode interval is between 250 us and 4 ms.

An electrosurgical generator for supplying radio frequency (RF) power to an electrosurgical instrument that has been introduced to the surgical site for cutting or vaporizing tissue immersed in a saline medium is disclosed, with a maximum peak RF voltage of less than 500V and a maximum root mean square voltage of less than 360 Vrms wherein the electrosurgical generator RF transistors are configured as an H bridge circuit comprised of 2 half bridge pairs of transistors with a maximum output current in excess of 3A root mean square with an RF current measurement sensor coupled to a control circuit able to within ½ of the RF cycle, alter the switching pattern of the RF transistors such that the voltage difference between the centre point nodes of the 2 half bridge pairs remains substantially zero, but the impedance between centre point nodes via 2 of the 4 switching transistors remains less than 1 Ohm.

An electrosurgical generator for supplying radio frequency (RF) power to an electrosurgical instrument that has been introduced to the surgical site for cutting or vaporizing tissue is disclosed, the electrosurgical instrument comprising at least 2 poles, the RF output of the generator being coupled to the electrosurgical instrument by at least 2 conductors, the generator comprising: a series coupling capacitance between the RF source and the connections to the electrosurgical instrument a means of measurement of the polarity of dc bias appearing between the poles of the electrosurgical instrument a means of disabling the RF output in response to one or more adverse polarities between the poles of the electrosurgical instrument.

An electrosurgical generator for supplying radio frequency (RF) power to an electrosurgical instrument that has been introduced to the surgical site for cutting or vaporizing tissue, the electrosurgical instrument comprising at least 2 poles is disclosed, the RF output of the generator being coupled to the electrosurgical instrument by at least 2 conductors, the generator comprising a series coupling capacitance between the RF source and the connections to the electrosurgical instrument, a means of measurement of the polarity of dc bias appearing between the poles of the electrosurgical instrument, and a means of annunciating an alarm in response to one or more adverse polarities between the poles of the electrosurgical instrument.

In certain implementations the adverse polarity is defined a positive voltage at the active pole or poles relative to the return pole or poles of the electrosurgical instrument.

In certain implementations the adverse polarity is defined a negative voltage at the active pole or poles relative to the return pole or poles of the electrosurgical instrument.

In certain implementations the adverse polarity is defined a change in polarity during RF activation of the voltage at the active pole or poles relative to the return pole or poles of the electrosurgical instrument.

An electrosurgical generator for supplying radio frequency (RF) power to an electrosurgical instrument that has been introduced to the surgical site for cutting or vaporizing tissue immersed in a saline medium, with a normal cutting or vaporizing interval with maximum peak RF voltage of less than 500V and a maximum root mean square voltage of less than 360 Vrms with a preamble interval following initial RF activation and preceding normal cutting or vaporization with a diagnostic voltage of less than 180 Vrms during the preamble interval wherein impedances measured during the preamble interval should be both greater than a lower limit and less than an upper limit to allow commencement of the normal cutting or vaporizing interval.

In certain implementations the lower limit is not greater than 20 Ohms.

In certain implementations the upper limit is not less than 290 Ohms.

This summary is not intended to be limiting of the invention. The invention is further described in the following detailed description and claims.

### FIGURES

The subject matter described herein can be illustrated by way of exemplar embodiments and the following figures:

FIG. 1: Treatment System.

FIG. 2: Generator outline (shows overall generator electronic architecture).

FIG. 3: Incumbent Impedance and Power Time Traces at Fire-up (showing how a loop electrode fails to get past a bubble generation stage).

FIG. 4: Impedance and Power Time Traces at Fire-up (for system with power surge).

FIG. 5: Power Surge Fire-up Control Algorithm.

FIG. 6A: Sheath and Telescope with Electrode Deployed.

FIG. 6B: Sheath and Telescope with Electrode Retracted.

FIG. 7: Detail Architecture for Peak Current Limiting Embodiment within Generator.

FIG. 8: Hemostatic Impedance and Power Time Traces (timeline).

FIG. 9: Hemostatic Waveform Control Algorithm.

FIG. 10A: DC Polarity Detection Circuit.

FIG. 10B: DC Bias Voltage Waveform (RF offset) and Detection Output Signals Time Traces.

### DETAILED DESCRIPTION

These disclosures relate to optimisation of electrosurgery in a saline field as used for volumetric reduction or removal of tissue associated with the prostate gland, bladder cancer or uterine pathologies such as polyps or fibroids. Bipolar electrosurgery is generally distinguished from monopolar electrosurgery by the requirement to dissipate less than 1% of the delivered power in an electrical pathway that does not involve the active accessory conductors. This is a safety assessment made using a model of the human body specified in the electrosurgical device Particular Standard under IEC 60601-1.

During saline field bipolar electrosurgery Normal saline, specified at 9 g NaCl per liter of H₂0, is flushed across the site of resection via a cannula and provides several benefits.

Firstly, it serves the purpose of clearing resection specimen debris and blood from the surgical field, which maintains the clear field of view required by the surgeon to operate safely and efficaciously, without the patient risks associated with irrigants used in standard monopolar electrosurgery due to saline being isotonic and leading to reduced complications and faster recovery times.

Second is the benefit of the saline irrigant heat capacity providing a constraint against non-reversible thermal damage to tissue away from the point of contact. This thermal energy hazard arises as waste energy from the electrosurgical effect at the active pole of the bipolar electrode and is increased with the delivery of higher levels of power. Through increased irrigant flow this hazard, along with that of reduced visibility mentioned previously, is mitigated.

A third benefit is that saline provides a conductive medium within which to position the return pole of a bipolar electrode without requiring tissue contact to complete the RF electrical circuit at the distal end of the bipolar electrode. Soft tissue conductivity is in the range 0.1 to 0.65/m depending on intracellular electrolyte content; normal saline at circa 1.65/m is comparatively conductive relative to tissue and this conductivity increases with temperature. Consequently, immersing the active and return poles of the bipolar electrode in the saline medium tends to constrain the divergence of the RF electrical field, compared to a similar arrangement without saline.

In physics, a higher divergence of a field is associated with the spreading out of that field. As a result of this constrained divergence it is possible to increase the separation between active and return poles of the active accessory while still maintaining a bipolar configuration. The RF thermal dissipation distribution pattern is also predominantly constrained local to highest density RF current flux lines and these remain within the saline medium, close to the line between the active and return poles of the active electrode, irrespective of contact with tissue and providing electrosurgical effect with improved precision and patient safety.

By example, a bipolar saline loop electrode might have a separation of 5-15 mm between active and return poles and still remain effective with the return electrode only in contact with the saline medium and not in contact with tissue. This separation is attractive as it allows a bipolar electrode to deliver a coagulation or cutting tissue effect at a single point of metal contact to the electrode. This is a utility similar to that readily achieved with a monopolar system, but without the hazards of remote thermal injury. The saline conductive pathway between electrode poles is also self-enhancing, as the local saline differentially becomes more conductive as ohmic heating locally heats saline passing there between.

Given the stated surgical benefits of using saline as an irrigant, and its resulting reduction in patient complications and recovery times, it is understandable that bipolar electrosurgery in saline has gained favour with surgical teams and in teaching institutions since its introduction.

With respect to generic saline field bipolar electrode designs, there are resecting active loops designed to efficiently remove ‘chips’ of tissue large enough to allow examination by a pathologist. This also represents the faster method of tissue removal, perhaps at the expense of the risk of abrupt transection of local blood vessels, and increased bleeding. The alternative saline field electrode design is a tissue vaporizing type, which has a comparatively large surface area with active and return poles placed closer together than for the loop electrode. The large surface area active electrode, when brought into tissue contact, transforms the soft tissue into a fine suspension in the saline medium. This is continually flushed away, requires a greater overall amount of energy compared to the loop electrode for removal of the same amount of tissue, and so on a power-constrained RF energy platform, results in a lower tissue removal and debulking rate. The rationale for greater energy requirement is that all removed tissue typically passes through the plasma that envelops the active pole when brought within 0.5 mm of making contact with tissue while a suitably high RF voltage is applied. By contrast to a vaporizing electrode, only the surface of each of the chips of tissue removed by a loop electrode should be vaporized in the plasma at the electrode active pole.

The benefit of the vaporizing electrode is that surgeons are less likely to get into difficulty with excessive patient bleeding, as the vaporization frontier advances slowly enough to maintain a higher level of haemostasis from the associated thermal margin ahead of the vaporized boundary. Due to its reduced tissue removal rate and large active electrode, it is also much harder for the surgeon to cause unintended injury such as a perforation. For this reason, it is also safer to place a vaporizing electrode in contact with tissue before activating RF intended to generate a cutting plasma.

By contrast, best practice with a loop electrode requires a cutting plasma to be established around the active, cutting loop, pole of the bipolar electrode, while it is just out of contact with tissue, in free saline. This is because there is a risk of the surgeon unwittingly removing or transecting more tissue than intended due to a combination of a lack of triangulation based depth perception of the active pole tip position relative to tissue; the loss of tactile feedback through the introducing cannula, and the sudden transition of the active pole tip from a passive wire with a solid boundary resting against solid tissue to a plasma enveloped volume capable of resection under the lightest of pressure contact with tissue.

Regardless of method, resection or vaporization, establishment of a plasma around the active electrode in a rapid and consistent manner across all conditions is critical to safety and efficacy. Without this the effects are longer or incomplete procedures, along with patient injuries and complications.

Due to the high conductivity of saline as already discussed, an immersed bipolar electrode will have a low impedance between active and return poles. A plasma will only develop once there is a high enough voltage gradient adjacent to the active pole, requiring an applied RF voltage of the order of 250 Vrms between poles. Colloquially this is called ‘fire-up’ of the active pole.

By design, the electrical path impedance local to the active pole is typically designed to be higher than that at any other point in the saline medium, including that adjacent to the return pole. For a vaporizing electrode this is enhanced by placing the active pole against tissue, which is of a lower conductivity as discussed earlier.

Fire-up at the active pole, is achieved by rapid local heating of saline in contact with the active pole sufficient to vaporize the saline in contact with the entire surface of the pole. For the purpose of this analysis, a non-ionized vapor medium enveloping the surface of the pole is an electrically insulating layer, and so as electrical contact between the pole and saline is lost, the entire applied RF voltage abruptly appears across the establishing vapor gap. This is observed as an abrupt change in the gross electrical impedance between electrode poles.

The locally increased electric field initially draws an arc as soon as the field strength increases to the breakdown value of the vapor. The local temperature-rise and charged species produced by this breakdown strip some vapor atoms of their electrons resulting in an incandescent conductive plasma between the pole surface and the saline. The incandescence arises from recombination of electrons with atoms and the signature fall in energy states associated with the species of atoms defining the spectral emission. For a loop electrode the active pole wire is also observed to glow red indicating a temperature in excess of 600° C.

There is an energy equilibrium established where almost all the applied RF power converted into this plasma volume is transferred away at the saline boundary to the plasma. Any reduction in temperature of the plasma as a bulk increases its impedance increasing the concentration of the applied power to this region of the electrical pathway between poles. Over a particular range of applied power, the plasma impedance is thus self-modulated to maintain maximum power density within the plasma medium, and the power density in the saline medium arising from ohmic heating is substantially reduced. By example, an unfired-up 4 mm loop electrode used for the TURP (trans-urethral resection of the prostate) procedure may have 22 Ohms impedance between poles when immersed in saline, but dissipate 100 W of RF power when fired-up with an applied RF voltage of 300 Vrms. This fired-up operating point corresponds to an impedance of 900 Ohms, with the increase in impedance over that for the unfired-up state being due to the film of plasma-filled vapor inserted in the electrical pathway between the active pole and the saline bulk. The informed reader will appreciate that excluding the RF power dissipated in the plasma, this reflects a 40:1 drop in dissipation due to ohmic loss in the saline medium at a given applied RF voltage.

Where a plasma collapses in one region of the active pole surface, the resistance of that local path is reduced, increasing the proportion of the RF power applied being absorbed locally. This local increase in power density corrects for the decrease the vapor gap locally until it near-matches the average. Similarly, if there is an increase or decrease in applied RF power, the vapor gap increases or decreases, with a corresponding change in aggressiveness when used for cutting soft tissues.

Due to the continual thermal transfer to saline at the vapor surface, a given minimum amount of RF power is required to sustain the plasma at the vapor gap, and below this power level, local partial collapses in the vapor gap result in a collapse over the whole vapor volume. Where this occurs, the impedance between poles falls by over an order of magnitude, with commensurate redistribution of the applied RF power away from the active pole. This represents the loss of the fired-up state. The minimum power required to sustain the fire-up plasma state around a device's active electrode can be very dependent on the temperature and flow rate of saline flush being employed.

While it is entirely feasible to design electrosurgical systems with higher power ratings, the preponderance of electrosurgical systems currently marketed for saline field electrosurgery are unable to fire-up a 4 mm loop electrode in free saline, at the flow rates discussed previously and preferred by surgical teams. This inability to fire-up such electrodes is due to a failure to achieve a high enough power density around the electrode active loop, necessary for the formation of a complete vapor gap; the precursor to fire-up. Electrosurgical systems are generally rated to deliver less than 400 W, which equates to 94 Vrms for the saline-wetted 22 Ohm loop electrode previously described. Consequently, the surgeon is obliged to bring such a loop into deliberate and increased contact with tissue in order to achieve fire-up. This results in a substantial loss of control and precision, bringing out the hazards discussed earlier. Ironically one of the concerns over delivering more power to the loop is an insufficient speed of control to manage such higher power delivery transient and an associated lack of the effective risk controls to manage the burn hazards posed by higher RF power delivery to the patient.

Prior Art U.S. Pat. No. 7,717,910 discloses a method of allowing a loop electrode to transiently sustain a high power transient supplied from a capacitor energy reservoir, with the steady state output power limited to normal electrosurgical levels of less than 400 W range.

This solution is deficient in several respects and lacks refinements that are now possible. Specifically, the use of an energy reservoir to ride-through the fire-up power surge requirement requires a delay to charge up the capacitor bank. In a presently marketed embodiment this corresponds to an observed delay of about 150 ms before RF energy delivery to the loop can commence. When added to the possible 150 ms delay required to form a vapor gap around the 4 mm electrode active loop, the surgeon may have to tolerate an apparent response time of 300 ms from the point of recognition of the RF activation request by the electrosurgical generator.

To achieve the fire-up of a 22 Ohm 4 mm electrode, the delivered power may transiently be 3000 to 4000 W, but based on the topology disclosed, this would taper down to near 50% of that level at the end of the supportable power transient. The design disclosed is also unable to make use of almost 50% of the energy stored in its capacitor reservoir, as there is a minimum RF voltage of circa 250 Vrms below which a plasma will not be achieved. Note that the solution of increasing the reservoir size with the prior art solution would carry the penalty of extending the capacitor bank charge up time.

A further disadvantage of the prior art solution is that, based on empirical measurements by the applicant, the energy reservoir size proposed is undersized and in high saline flow conditions it is possible for fire-up to have not occurred at the end of the 150 ms power surge interval. The applicant has determined that a greater amount of energy is needed in the power surge required to fire-up a 4 mm loop under the combination of room temperature saline at high flow conditions. With the prior art device, the surgeon has to wait for repeated charge and surge discharge cycles from the electrosurgical system, adding to delay in fire-up, adversely affecting usability and encouraging placement of the loop adjacent to patient tissue to increase initial impedance.

It is also known that during bipolar electrosurgery procedures in saline a more hemostatic removal of tissue can be required due to patient anatomy and condition, or physician preference, to help keep the field of view clear enough. To accomplish this, current systems use a “modified” or “blended” output in an effort to slow down the tissue removal rate slightly, allowing addition coagulative effect to take place.

Prior art U.S. Pat. No. 7,195,627 discloses a hemostatic waveform intended for use with a power surge-capable electrosurgical system with an internal energy reservoir. Partly as a result of the ability to resect greater volumes of tissue with a loop electrode, it is possible for surgeons to transect multiple blood vessels in close succession and so a method was proposed to reduce cutting speed and increase haemostasis. In this prior art the hemostatic waveform was made active whenever there was detected to be an energy surplus in the internal energy reservoir. This was a method of repeatedly quenching the established plasma and so forcing repeated operation of the electrode in its purely dissipative, non-cutting mode. An improvement is proposed on this solution which recognises that the greatest dissipation is achieved by avoiding the OFF state of the pulsed waveform.

In addition, prior art U.S. Pat. No. 7,211,081 discloses a method of avoiding excessive RF peak currents by requiring the RF devices to be switched off in such an event. These excessive currents, which can be associated with system and equipment damage, and an increased patient harm, are an inherent risk of electrosurgery that requires elimination or mitigation. Alternative methods are disclosed here, each with advantages over the previous disclosure.

The surface area of the electrode return pole in contact with saline is normally substantially greater than that at the electrode active loop, which ensures fire-up preferentially occurs at the active. During RF activation however, electrode design and surgical environment characteristics can reverse this. As an example, bubbles that form at the electrode active loop and can collect on the superior surface of the prostatic capsule at the distal end of the cannula. This may cause a reduction in the surface area of the return pole immersed in saline to an extent that results in the fire-up at the return pole instead of the loop. Fire-up away from the active electrode is hazardous because it is unexpected and also out of sight of the telescope inserted to provide a view of the surgical site. This is a known cause of an accidental patient harm such as perforation of the prostatic capsule or bladder, and sphincter injury. A means is disclosed of detecting such an adverse switch between which electrode pole is fired-up.

As is known to those skilled in the art, software to control safety critical equipment cannot be operated on operating system platforms targeted at general purpose computing, such as the Personal Computer (PC). This is due to the hazard of safety-critical operation prospectively being affected by Software Of Unknown Provenance (SOUP). Instead microprocessors are typically combined with other control related peripheral hardware circuits, such as timers, serial ports and analog converters in various microcontroller offerings. Microcontrollers can be targeted at specific applications by inclusion of particular peripherals and as such are considered embedded or customised to the specific application.

Typically, software code is exclusively developed for the safety-critical medical device application, and excludes the use of an Operating System (OS) in the interests of speed and reduction in complexity. An example of a microcontroller range widely used in embedded applications is the STM32 series which includes 32 bit microprocessors designed by ARM Holdings. These will support fast computational loops, and adjust repeatedly revised analog power demand levels based on analog sensory feedback. In the prior art example of a peak power surge of 4000 W the time available for control could be considered to be 40 times more limited than that needed to assure control of a 100 W output, purely on a basis that patient injury is related to unintended energy delivery.

A risk assessment should also require that the failure of the software control loop to operate be detected in as short an interval as is necessary to avoid significant injury to the patient from unintended energy delivery. This is typically referred to as a watchdog circuit, which should have sufficient timing margins to avoid nuisance intervention of normal microcontroller operation.

Operating a control loop for a 4000 W output at 50 us (micro second) intervals corresponds to a control adjustment every 0.4 J, and setting the watchdog trip interval at 4 control loop periods (200 us) we could have up to 1.6 J of uncontrolled delivery in the event of a microcontroller failure. As a yardstick 1.6 J could elevate a 1/60 mL micro-drop of water by about 20° C., which is considered a non-injurious severity. A further benefit of such a fast control loop, is that the rapid impedance changes associated with the turbulent formation of the vapor gap is less likely to cause aliasing of measurements and incorrect control adjustments. The vapor gap typically forms in discrete bubbles which join up during the fire-up, but these bubbles can collapse very rapidly, for example in less than 1 ms, producing a kettle-type audio noise in the process.

Within each control interval repeated every 50 us during RF cut activation the control algorithm is likely to sample the RF output power, RF output current, the RF output ac voltage, and the RF dc bias voltage. The electrode tip impedance is deduced from the ratio of RF output current and RF output ac voltage.

The impedance is then compared against allowable limits depending on the stage of fire-up of the electrode. While the output voltage is low, the impedance is preferably in the range 20 to 300 Ohms, rising through to 800-1000 Ohms within 1 second.

The RF power may be deduced from known power flow from the dc supply coupled to the RF stage, allowing for RF converter losses; or may be empirically mapped in 2 dimensions from relationships between RF ac voltage, RF current and delivered power.

Each of the output RF power, RF ac voltage and RF current measured parameters are compared against independent parameter limits, which may be time profiled, for instance to allow for a power surge at start up. At each 50 us control point, the power launched from the dc supply coupled to the RF switching stage is only increased if none of the parameter limits is exceeded, in which case it can be incremented. Such a three parameter closed loop control for incrementing or decrementing the RF output allows deterministic compliance with published output power characteristics expected for electrosurgical systems. These are commonly published in accompanying documentation for the generator and define the expected RF delivery characteristics for the generator at a given user setting, anticipating a range of tip impedances typically from 20-1000 Ohms. What is distinctive for this implementation is the high rate of adjustment of control, a need arising from the prospective high power RF output couple from an AC mains supply with an equally high power continuously rating. On lower power systems control or intervention computation intervals of the order of 1 ms to 10 ms are common.

The advantage of such a frequent measurement and control update rate is that the software algorithm can be made sophisticated such as to adopt states of behaviour representative of transient conditions related to:
a fully wetted but rapidly warming up electrode active loop;
a tip with a developing vapor gap around the electrode active loop;
a partly-fired up electrode active loop;
a fired-up electrode active loop in stable equilibrium;
a faulty electrode,
an electrode return pole situated in a bubble; or
an electrode active loop engaged with tissue.

These deductions may be based on the previous detected state, states since fire-up and present and recent combinations of values for RF impedance, RF power, RF ac voltage and output dc bias.

A particular control required for this design of electrosurgical generator with a high power throughput capability of approximately 1000 W, is a monitor to ensure that the delivery power is being utilised to establish a plasma, and to ensure that only the energy dose expected for this purpose is delivered.

As such a tally is maintained for the first 100-200 ms after start up, where the RF power measured at each 50 us control interval is used to ensure that the maximum energy delivery is limited to 100 J for a saline field electrode with a 4 mm loop. Other limits could in principle apply for other size instruments.

By way of example in addition to the hazard detection interlocks already described, for a saline field electrode with a 4 mm loop the following sequence would be required for RF delivery to continue after initial RF cut activation: For 0-100 ms, no more than 100 J may be delivered;

After 200 ms the tip impedance has to have exceeded 300 Ohms

At 300 ms the power limit is dropped to the user set average power limit, typically at the lower end of the range of 100-300 W;

After 1 s of RF activation the impedance is above 600 Ohms.

The tip impedance at point of RF activation is likely to inform whether the tip is engaged in tissue or is in free saline, and if in free saline, the ratio between initial loop impedance and the minimum loop impedance is likely to inform on the change in saline temperature before fire up, and thus the temperature of the saline flush, which in turn should indicate what maximum fraction of 100 J may be required to complete fire-up, and the maximum average power needed to sustain the plasma in the vapor gap against thermal losses to the saline flush.

As a general safety principle, best practice is to use the minimum waveform intensity needed to achieve a desired treatment effect. To this end after a 1 second stabilisation interval, the output power could be reduced below the user set average power limit and then regulated in closed loop fashion to achieve an impedance that was above measured in the partial plasma state of the first 200 ms interval and up to 20% below the maximum observed in the since establishment of the RF plasma. This is so as to minimise the cut-aggressiveness of the plasma without extinguishing it. This control loop adjustment could operate on a fast power increase; slow power decrease basis, so as to avoid vapor gap collapse at the end of each tissue resection stroke. A further benefit arising would be that cutting would be more haemostatic, due to reduced cut aggressiveness, and yet would not stall.

For an even more hemostatic cut waveform a surgeon-selectable pulsed regime could be employed where the RF voltage limit was amplitude modulated between a plasma-cut level of the order of 300 Vrms and a plasma-quenched voltage level of the order of 120 Vrms. The depth of modulation for this purpose would be nominally 60% but at least between 40% and 75%. An example embodiment would be to have the envelope modulation frequency of approaching 1 kHz (1 ms period), with a shorter interval at the lower amplitude level than at the cut amplitude level, with the mechanism only coming into operation while the tip impedance indicated significant tissue engagement, a condition that is potential precursor to excessive bleeding. Tissue engagement extent is determined by a combination of lower power consumption and high tip impedance. The lower power consumption arises as a result of the comparatively high electrical resistivity and high thermal resistivity of tissue compared to saline.

In addition to the additional dissipation during the plasma-quenched portions of the waveform offering an improvement in haemostasis over the 100% modulation solution proposed in the prior art, the presence of a lower amplitude RF voltage during the plasma-quenched state can be utilised for diagnostic purposes.

During RF cutting in a saline field, impedances in excess of 1000 Ohms and less than 80 W consumption are typically associated with a well-established plasma with the cutting loop well engaged with tissue, with low impedances in the range 600 to 900 Ohms with greater than 100 W dissipation being typical of more conservative and safer engagement of the cutting loop with tissue. For RF waveforms of the order of 120 Vrms the absence of a vapor gap or plasma enveloping the active pole of the loop electrode mean that impedances are likely to be between 15 and 300 Ohms with varying degrees of engagement between tissue and loop. Accordingly, it is possible to devise impedance measurement based rules for commencing an interrupted cutting waveform, and for transitioning back from the quenched state into the cutting state.

Means of detecting the adverse fire-up of the return pole are disclosed. The surgeon is trained to maintain visual contact with the electrode active loop whenever RF is activated. Also required practice is the issuance of a distinctive audible tone by the generator once RF is activated for cutting tissue.

A first means relies on the generator being able to detect when an RF cutting plasma has been detected. This can be deduced from the impedance measured between active and return poles of the electrode during activation. By way of example for an electrode with a 4 mm sized loop, an impedance in excess of 900 Ohms at an applied RF voltage of 300 Vrms is indicative of a well-established plasma, and thereafter the impedance should not fall below 600 Ohms. While this tip impedance requirement is met, the generator may vary the audible tone issued during cutting as a confirmation of the presence of a plasma. This alerts the surgeon that there should be a visible plasma at the loop, the absence of which requires immediate attention, most likely cessation of RF activation and flushing of vapour from the prostatic capsule occurred from aggregated bubbles released from the saline at point of contact with the electrode active loop plasma.

Prior art U.S. Pat. No. 6,547,786 discloses the concept of relating the extent of aggressiveness of an electrode tip plasma, to the amplitude of the dc voltage appearing at the active RF pole relative to the return RF pole, arising from rectification said active electrode plasma. A non-polarised RF ac supply from the generator is coupled to the tip by a series capacitance intended to block any dc current path through the patient. What is observed is a dc voltage between the active and return poles, which is not applied by the generator. This is superimposed upon the applied non-polarised RF voltage. The inventors disclose a use of the polarity of dc voltage to allow indication of which of the 2 RF poles is fired-up, or an indication of a change in which pole is fired-up. A negative bias of between 10 and 200 Vdc is expected at high impedance at the active electrode pole relative to the return electrode pole during the normally fired-up state. The dc bias is measured by the generator using a high impedance connection to the patient connections which is then low-pass filtered to allow measurement of any dc bias. Either the polarity of the voltage once above a chosen threshold, or the actual dc voltage can be signalled to the system microprocessor, allowing for software controlled issuance of alarms with possible software-controlled interruption of the RF waveform until the RF activation switch is first de-activated and then re-activated.

As the amplitude of the dc bias is known to be linked to the aggressiveness of the plasma, the threshold at which dc bias detection is significant may vary, and it may be desirable to either digitally filter the dc bias signal or vary the bias detection threshold according to the actual RF power and voltage amplitude. This sophistication in discrimination of bias is easier to implement if the dc bias voltage is measured on a scale, for example by being couple to the analog to digital converter of a microcontroller for repeated sampling and processing during RF cut activation. This allows information on the profile of dc bias to be correlated against the delivered RF power for maximum sensitivity of detection of which electrode pole is fired-up, or to detect a change in which electrode pole is fired-up.

As an improvement over the prior art, which utilizes an energy reservoir to fire-up the electrode active loop with a tapering power output starting at up to 4000 W, a 1000 W RF source is used instead. In the preferred embodiment the electrosurgical generator including its mains to dc power supply are rated to draw this transient power surge from the incoming mains supply.

The applicant has empirically determined that a 1000 W RF source is able to deliver up to 100 J quickly enough to ensure formation of a vapor gap, and fire up of the loop. The reduction in peak power is also a 4:1 improvement in terms of the unintended damage possible to the telescope inserted through the cannula to observe the active tip function. Mains to dc supplies, compliant with international medical device safety requirements are available, one such suitable solution being the use of 2 universal mains input MCB600 600 W units from ROAL Electronics. These can be operated in tandem to deliver the surge required.

Protection of the output from excessive current levels. The purpose of limiting the output current level is primarily to prevent accidental arc damage to third party medical instrumentation, including the rod telescope and working sheath. The electronic power topology capable of the greatest capacity with a given rating of power transistor, is the H-bridge circuit, familiar to those skilled in the art. This is formed of 4 identical transistors in 2 series or half-bridge legs connected across a dc supply. The RF output is taken between each of the half bridge centre points which nominally switch at the desired output frequency in antiphase, so as to produce a square RF waveform with a peak to peak voltage amplitude of twice that of the dc supply. The U.S. Pat. No. 7,211,081 prior art solution is to halt the switching of these transistors in the event of a peak current excursion. This causes the H-bridge transistors to have to perform one otherwise unscheduled switching event and at maximum current amplitude. In sizing the H bridge transistors, the thermal limitations on their use arise from the Ohmic conduction losses associated with the RF output current, and more significantly at RF frequencies, the pulse power loss associated with transitions between transistor ON and OFF states. This can be reduced by avoiding switching transistors while there is an appreciable RF current flowing. This is normally achievable as the RF output stage can be tuned for minimal capacitive-lead current. The unscheduled additional switch required by this prior art solution therefore can place exceptional stress on the H bridge power transistors.

Using a well-designed current sensing transformer positioned in the RF output path from the electrosurgical generator, it is possible to faithfully reproduce the instantaneous wave shape of RF output current. This signal is then actively rectified using a standard active rectifier circuit but using video bandwidth operational amplifiers. With this approach any peak excursion is followed in the amplifier output to with less than 200 ns delay. As such for a 400 kHz (2.5 us period) RF waveform, the excess output current detection time is less than 1/10^{th} of a cycle.

The RF stage switching transistors can be economically sized to rely on the current limiting afforded by the upstream dc/dc power controller. However, where a surgeon accidentally activates the electrode with both poles still inside the cannula, up to 8000 W peak power where the RF cycle average power is 4000 W, can be thermally transferred in a metal vaporizing arc to the third party instrumentation, most commonly the telescope rod. With perfect alignment the telescope is kept out of contact with at least the electrode active loop. Misalignment is common as a result of tolerance build up between electrode, cannula and telescope positioning, and as a result of slight bending of the malleable electrode during use or insertion.

During an arc event, prompt intervention is required to avoid pitting of the metal surfaces of the third party instrumentation. The flare of such an arc is likely to also cause transient saturation of the digitally processed image captured by the surgical video system, which startles the surgeon and may at worst result in involuntary movement and at least result in concern over safety. A reduction of the cycle average power to 1000 W coupled to prompt interruption of the RF current under excessive current amplitude transients reduces these risks significantly.

It is worth noting that such an unwanted arc may arise from proximity between the electrode active loop and the third party metal instrumentation, rather than direct metal to metal contact, as the RF voltage can ionise the separating surfaces or air gaps if sufficiently short.

In a first disclosure related to current protection, the applicant identifies that it is preferable to insert a series transistor in the dc supply to the RF stage H bridge circuit, and to open this transistor's conduction channel (switch OFF) in the event of such an excessive current event. Using timing extension circuits, this dc supply can be instantly removed from the H bridge input, and returned at a suitable delay period later, typically several RF cycles. The advantage of this approach is that the H bridge devices may be more closely matched to the power requirement and this single device can be driven from a low power, simpler circuit as it switches infrequently. Including the time taken to open the dc supply gating transistor conduction channel, the overall time taken to interrupt the output RF current in the event of an excess current event is expected to be 200 ns. This is in addition to the delay in synthesising the full wave rectified current sensor signal, and so the delay in interruption of the RF current is in total 400 ns or 115^{th} of an RF cycle at 500 kHz.

In a second disclosure related to current protection, the applicant identifies that an alternative solution to that of the prior art, relies on the phase of the two RF stage half bridge legs being switched antiphase with respect to each other under normal operation, but immediately switched IN phase with each other during an excessive current event. In the prior art solution, the impact of switching OFF the RF stage devices is to apply a reverse polarity voltage across the output filter components until the current decays to zero. This is more important where the excess current detection time has allowed the current climb to a higher value than is desirable. Where the excess current detection time is short enough, it is possible to lower the trip level to just above normal operating conditions, and in such a scenario, it might be preferable to KEEP the current flow at this limit level but to not allow an excursion above the limit.

Switching the half bridge legs in phase with each other applies zero voltage to the output filter stage and so will not cause a reduction in the output current to the same extent as the prior art. In practice, energy transfer to the tip and circuit losses will allow a gradual decay of current. Switching phase difference can be returned to normal/antiphase at a desired delay period after the current had been detected to have fallen below the limit level.

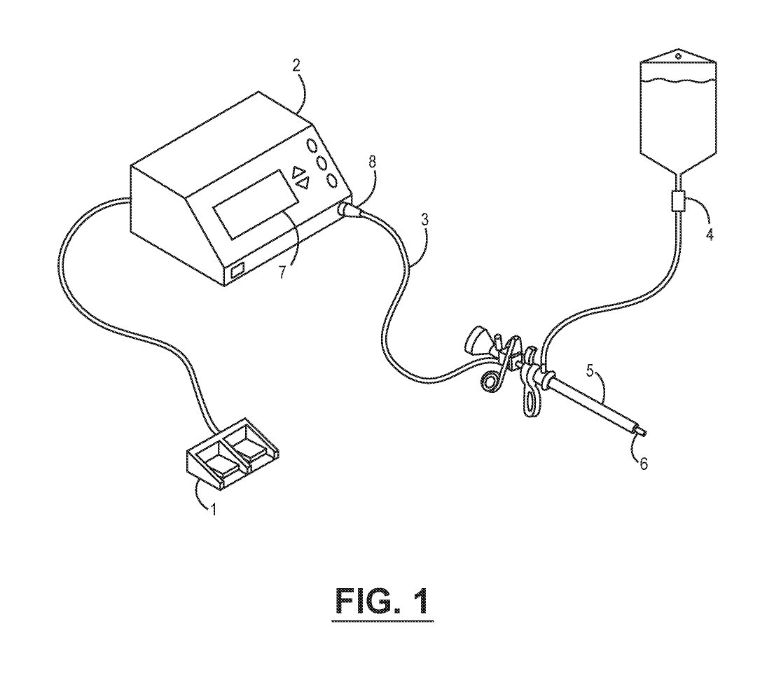

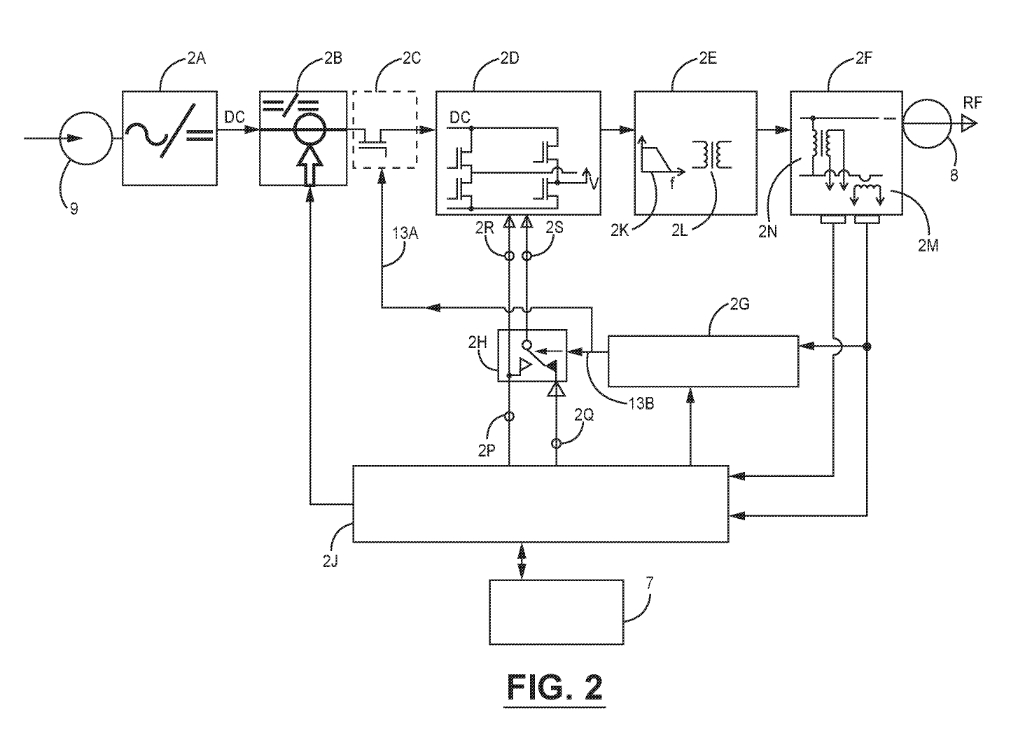

FIG. 1 depicts a typical endoscopic treatment system for the Trans-Urethral Resection of the Prostate (TURP), which immerses the electrode 6 in a conductive Normal saline medium. The system is comprised of a footswitch assembly 1, primarily intended for allowing the surgeon to electively activate the RF treatment output waveforms from the electrosurgical generator 2 without contamination to the surgeon's hands. The status of the RF electrosurgical generator is annunciated audibly using tone, and via display area 7 on the fascia of the electrosurgical generator. The RF output 8 from the electrosurgical generator is coupled to the electrode 6 via an interconnecting cable 3 which includes at least 2 conductors for the at least 2 RF output poles of the electrosurgical generator. The electrode 6 is inserted inside a sheath 5 through which is passed the Normal saline irrigant gravity-fed from a saline reservoir 4. The proximal end of the sheath 5 includes the objective end of a telescope and a lever system to actuate the axial deployment and retraction of the electrode 6 relative to the sheath 6. Prior to commencement of surgery the sheath is inserted towards the superior end of the urethra so as to position the distal end of the sheath at the enlarged prostate gland to be de-bulked by the cutting and vaporizing electrode 6. FIG. 2 illustrates the electronic architecture of the electrosurgical generator 2 designed to synthesize RF waveforms and provide a control and monitoring interface 7 for the surgeon. The mains supply 9 arrives into the system and is converted to a fixed regulated dc level by a medical-grade ac mains to dc power supply unit 2A. This can be a commodity unit as discussed in the preceding disclosures. A portion of the output power from the ac mains supply unit 2A is passed on towards an RF switching stage 2D by the variable output dc to dc converter power supply 2B. The impedance presented by the RF switching stage 2D is generally linearly proportional to the RF impedance between the poles of electrode 6. It will be apparent therefore that the voltage applied to the input RF switching stage is a function of the power throughput and the impedance between the poles of the electrode 6. The switching frequency of the RF stage 2D is in the range 100-1000 kHz, and preferably in the range 300-500 kHz. A series semiconductor switch 2C is an optional element and is typically embodied by a power MOSFET transistor. Under normal operation the switch is closed but under excessive peak current conditions the switch can be rapidly opened by a signal 13A from the output peak overcurrent detector 2G. The fraction of the output power from the ac mains power supply unit 2A that is coupled to the switching stage 2D is defined by a demand signal derived by the microcontroller 2J in response to a comparison of the RF output current, voltage and power to expected values given the current measured value for the impedance between the poles of the electrode 6. The status of the electrosurgical generator is audio-visually annunciated under control of the microcontroller 2J on the user interface which includes a display unit 7. The microprocessor 2J is also responsible for synthesizing the fundamental RF signal. 2 signals at the RF frequency but at 180° phase difference are supplied by the microprocessor 2J as signal inputs 2R and 2S to the RF switching stage 2D. In one embodiment in the event of an output peak overcurrent event, the output peak overcurrent detector 2G operates a semiconductor 2:1 multiplexor switch 2H via signal 13B which causes an immediate change in the phase difference between the signals 2R and 2S being coupled to the RF switching stage. Accordingly, under normal operation the RF stage 2D develops a square waveform output by using signal 2R and 2S as antiphase inputs for the 2 half bridge legs, and under peak overcurrent conditions, either the dc supply to the RF stage is removed by opening the series semiconductor switch 2C; or the phase difference between 2 half bridge legs of the RF switching stage 2D is immediately brought to zero under action of the 2:1 multiplexor switch 2H. During normal operation the square RF waveform is coupled through a band pass filter 2K and then a patient circuit isolating transformer 2L which also scales the output voltage appropriately for the desired RF cut waveform amplitude of up to circa 300V rms. The RF output leaving the composite filter and isolation stage 2E is generally sinusoidal at all RF impedances, which allows for simplification of the output metering process. The output current and voltage coupled to the patient via the RF output 8, are sampled by RF voltage sensor 2N and current sensor 2M. The signals from these sensors are then used to inform the control algorithms of the microprocessor 2J and the sensed output current signal is fed to the input of the peak overcurrent detector 2G.

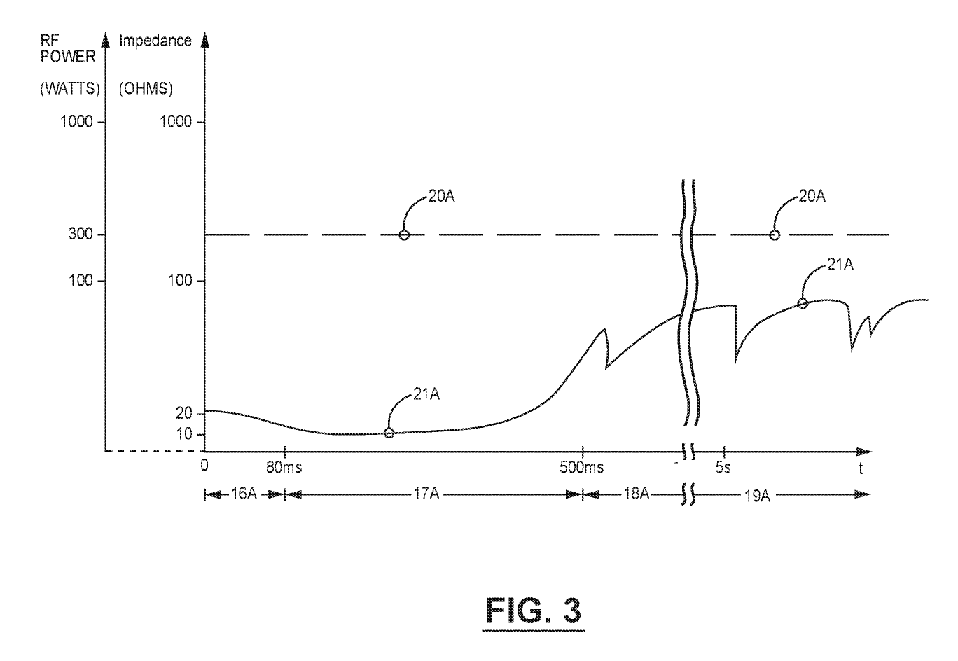

FIG. 3 illustrates why many incumbent generators that are reliant on a maximum RF power output of 200 to 400 W depending on model, fail to reliably fire-up a 4 mm loop electrode immersed in saline. The time traces of the RF power 20A coupled to the electrode and the resulting impedance 21A between poles of the electrode show they can fail to exceed 100 Ohms with a 300 W RF supply. In sequence, during an initial RF activation interval 16A, the impedance between active and return poles of the electrode is seen to fall from just over 20 Ohms to almost half that value due to an increase in conductivity of saline with an increase in temperature local to the electrode poles. During the second interval, 17A there is a crossover of competing opposite effects. A first effect is the increasing conductivity of the saline surrounding the electrode but the second is an increase in impedance at the surface of the electrode active loop due to the increasing formation of micro bubbles. During interval 18A these microbubbles aggregate and lose contact with the loop due to convection currents and buoyancy. Then they collapse abruptly as they make contact with cooler saline. This results in the start of a kettling and popping sound, but more critically results in an abrupt fall in the circuit impedance between the poles of the electrode, specifically because of the increased wetting of the electrode active loop pole. This cycle repeats with a cycle of slow build-ups in circuit impedance followed by rapid falls as further bubbles are released and collapse into the local saline. This is an oscillatory state the electrode remains in indefinitely represented by interval 19A, unless the electrode loop is partially masked by surgeon intervention, but this has associated hazards which are highlighted in the disclosure discussion.

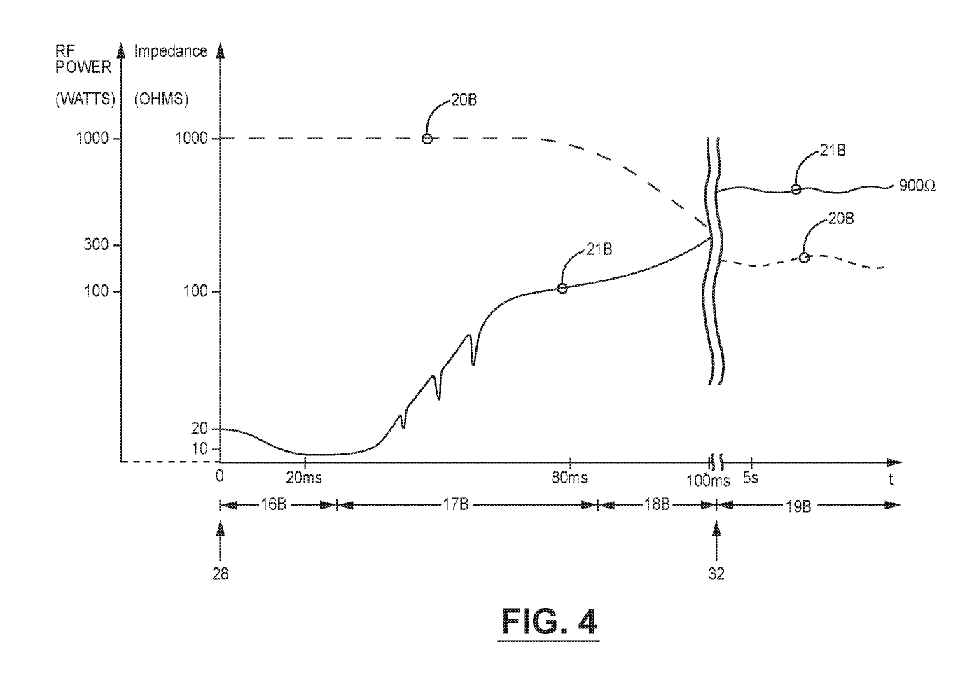

In FIG. 4 the marked benefits of a surge in the RF power 20B coupled to the electrode of approximately 1000 W for about 100 ms can be seen. In a first interval 16B starting at initial activation of RF at point 28, the impedance 21B between the electrode poles is observed to fall more rapidly than before due to the faster heating of saline. During the interval 17B the transition from a wetted electrode loop to one with a complete vapor gap occurs in circa 80 ms with fast recoveries from the now limited numbers of impedance collapses associated with released and collapsing bubbles. During interval 18B a plasma is starting to form but may not yet be in thermal equilibrium with the surrounding saline which is still heating up. As a consequence, although the power consumed at the electrode loop falls rapidly from 1000 W, at 100 ms from RF activation the impedance is still rising and the power is still significantly above the steady state consumption rate after circa 1 s. This steady state condition with a stable plasma characterizes interval 18B, and starts at point 32.

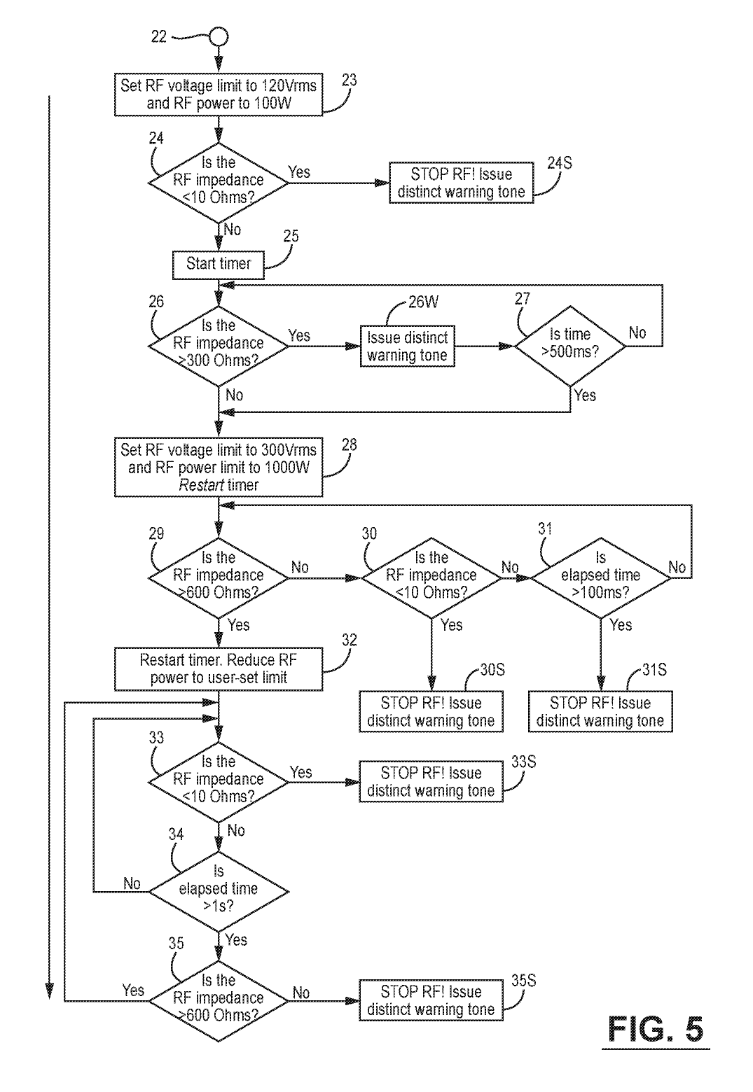

FIG. 5 is an example algorithm of decisions made and actions taken during the establishment of the plasma and during its steady state maintenance. Those skilled in the art of programming will appreciate that this is a simplified representation of the implementation of these controls and that this does not represent an optimal software coding architecture. The algorithm starts at 22, and at step 23 a nominal power level with a non-cutting RF voltage amplitude is set by the microcontroller. This is to allow impedance checks for electrical shorts or bubble collection around the electrode return pole. During this preamble interval the electrode impedance is repeatedly checked at step 24 for excessively low impedance. In the event of a short circuit or near short circuit being likely the software halts the RF activation process at step 24S. In this embodiment, the design is intended to also check for an impedance in excess of 300 Ohms at step 26 and to issue a warning at step 26W for at least half a second or until the impedance becomes acceptable. The intention is that this gives the surgeon enough time to investigate the problem and intervene by releasing the activation switch or the option to override the warning at step 27 by waiting for the half-second time out. At step 28 the RF output is set to a surge power level of 1000 W and the RF voltage limit is increased to that capable of eventually sustaining a plasma. The surge interval in this illustration is limited to 100 ms at step 31 and during this interval the impedance should not fall below 10 Ohms at step 30, indicative of a short circuit or near short circuit between the poles of the electrode. Such a short circuit results in the microcontroller halting RF delivery at step 30S. If the impedance between the RF poles is not measured to have exceed 600 Ohms at step 29 within 100 ms at step 31, the microcontroller halts RF delivery at step 31S. An impedance of greater than 600 Ohms is indicative of an establishing plasma, which means that almost all the power delivered to the electrode will now be dissipated into the volume immediately surrounding the electrode active loop. This results in a much lower power requirement to be delivered to the electrode in order to sustain the plasma, and so at step 32 the power limit is dropped to the value set by the user. This is typically in the range 100-200 W. Beyond this point in time, for the reasons given, the impedance between the poles of the electrode is expected to rise even though the power applied to the electrode is reduced. For the first second, checked at step 34, the only requirement is that the tip impedance is not indicative of a short circuit at step 33, but thereafter the impedance should be at least 600 Ohms, step 35, or the microcontroller will halt RF delivery at step 35S. The RF is also halted at step 33S if the impedance is indicative of a short circuit across the electrode at any time after the power has been dropped to the steady state limit at step 32.

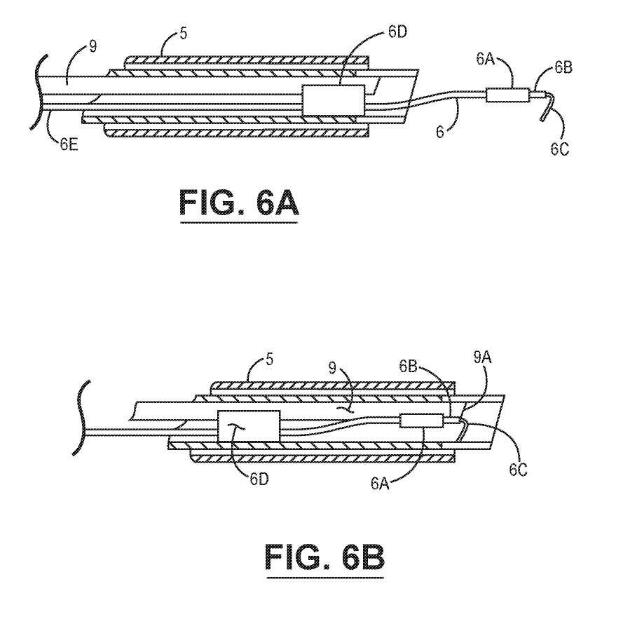

FIGS. 6A and 6B show the sheath 5, metal clad rod telescope 9, and the electrode 6 in first a deployed and then a retracted position. The electrode 6 is slidably attached to the telescope rod by an insulating polymer clasp 6D and is actuated longitudinally inside the sheath under control of the surgeon advancing and retracting the proximal end of the electrode shaft 6E. At the distal end of the electrode 6 there are 3 exposed metal areas. Distal to the polymer clasp, the electrode shaft divides into 2 identical yoke arms which part bilaterally and upwards from the shaft 6E. Along these arms are the 2 identical return poles 6A, which are separated from the distal active loop pole 6C by ceramic sleeve insulators 6B. Ceramic material is required for its refractory properties. In FIG. 6B it is possible to see how close both active and return poles 6A and 6C of the electrode necessarily come to the metal surface of the distal end of the telescope rod 9. This is a particularly sensitive point of the telescope as there are seals between the distal lens and the telescope cylinder enclosure 9A that if damaged will render the telescope non-functional. Additionally, flashes that occur next to the distal end of the telescope will be particularly well picked up on the surgical video system and cause maximum disturbance to visualisation of the surgical site.

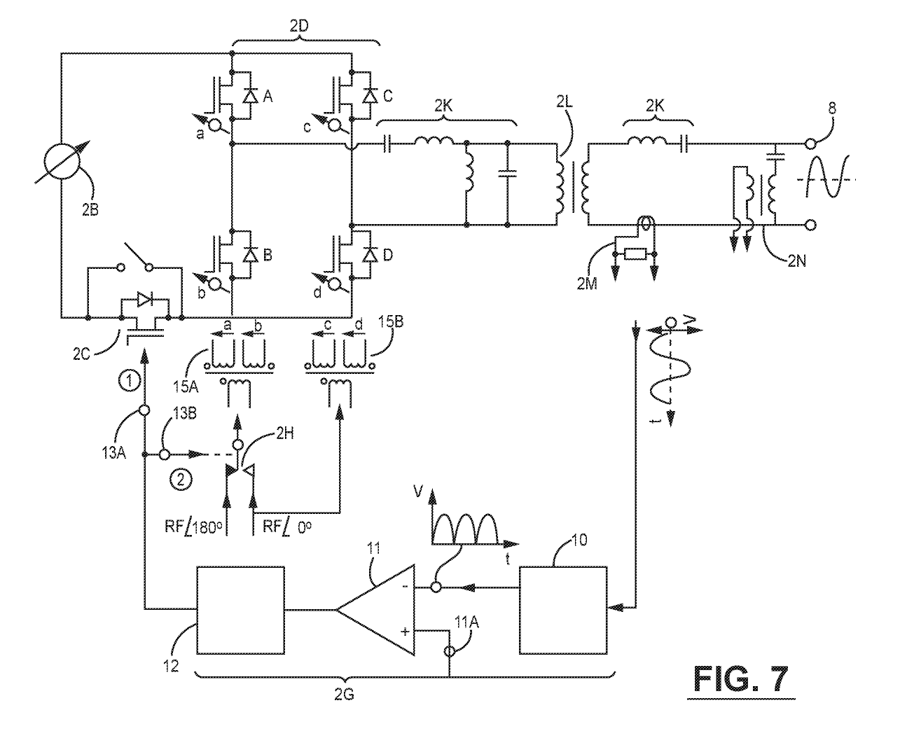

FIG. 7 shows more detail of 2 alternative embodiments for peak RF output overcurrent limiting outlined in FIG. 2. For reasons of clarity, information relating to the embodiment of the slower cycle-average closed loop control for RF output power, current and voltage is not shown. The variability in RF output amplitude that arises from this adjustment in the dc voltage coupled to RF switching stage 2D is represented by 2B as an adjustable voltage source. The RF switching stage 2D is comprised of 2 half bridges A,B and C,D respectively driven by gate drive signals a,b and c,d. Each pair of signals a,b and c,d is permanently an antiphase pair of square wave signals such that the centre node of each half bridge is either connected at low impedance to the positive dc supply coupled RF switching stage 2D; or to the zero volt potential. The signal pairs a,b and c,d are isolated secondary windings of the gate drive transformers 15A and 15B which are fed with square wave RF signals. The RF output current sensor 2M provides a low harmonic content sinewave to an active rectifier 10 which has a full wave rectifier output signal that is linearly proportional to the instantaneous amplitude of the RF output current. This is convenient as it allows a single dc signal level 11A to define the peak allowable output current. At this point the phase delay between the rectified signal and the RF source is less than 200 ns. A voltage comparator 11 which under normal running condition has a logic-high output, produces a logic-low output signal while the instantaneous output current is above the limit defined by 11A. This can be a very narrow logic-low interval especially if the RF output 8 is promptly interrupted. To independently define the length of time the RF output 8 is interrupted, the logic-low output transient from comparator 11 is pulse-extended by a triggered monostable circuit 12. The monostable circuit 12 includes a resistor-capacitor time constant that defines the actual length of interruption to the RF output 8. Depending on embodiment preference for implementation of a peak current limit, there is either a series MOSFET 2C between the dc supply 2B and the RF switching stage 2D; or the MOSFET 2C is replaced by a shorting link across the circuit nodes for the drain and source terminals of the MOSFET.

Where the MOSFET 2C is fitted, the normally logic-high output signal output 13A from the monostable pulse extender 12 ensures a low impedance ohmic connection between the output of dc supply 2B and the RF switching stage 2D. Immediately following a peak overcurrent event, the signal 13A transitions to a logic-low level for a minimum duration irrespective of the actual duration of RF output peak overcurrent. This logic-low level interrupts the forward power flow of current in the zero-volt connection between the dc supply 2B and the RF switching stage 2D. The overall latency between and RF output 8 overcurrent event and interruption of the operation of the RF switching stage 2D is typically 400 ns. This is less than ⅙^{th} of an RF cycle at 400 kHz. Typically, the RF is disabled for several tens of RF cycles to ensure that there is complete decay of the RF output 8 current and to bring the average thermal stress caused to no more than occurs under normal maximum load conditions. For this embodiment the gate drive transformers are fed with antiphase square RF signals at all times, equivalent to the 2:1 multiplexor 2H being permanently connected in the Normally closed position, depicted conventionally by the darkened triangle flag. The reader will appreciate that for this embodiment the 2:1 multiplexor 2H is not required and what is important is that the transformers 15A and 15B are driven in opposite phase to each other.

For the embodiment where the MOSFET 2C is replaced by a shorting link across the drain and source nodes, the logic-low output 13B of the monostable pulse extender 12 following a peak RF output overcurrent event is used to switch the phase of the input signal to gate drive transformer 15A from being antiphase with respect to the input signal to gate drive transformer 15B, to both signal having the same phase. While the output 13B of the monostable pulse extender 12 remains logic-low, the voltage between centre nodes of the 2 half bridges A,B and C,D of the RF switching stage 2D remains zero and at low output impedance.

For either embodiment of peak RF output overcurrent control, during normal operation the band pass components 2K are used to filter harmonics out of the variable amplitude square wave output from the RF switching stage 2D. The RF output voltage is also scaled as required for function, and isolated by RF transformer 2L. In addition to the RF output 8 current sensor 2M there is also an RF output 8 voltage sensor 2N close to the RF output 8. Following a minimum period after a peak RF output overcurrent event, the removal of the input voltage to the filter stage 2K results in interruption of the RF output 8 for a minimum period of time, a duration at the discretion of the designer.

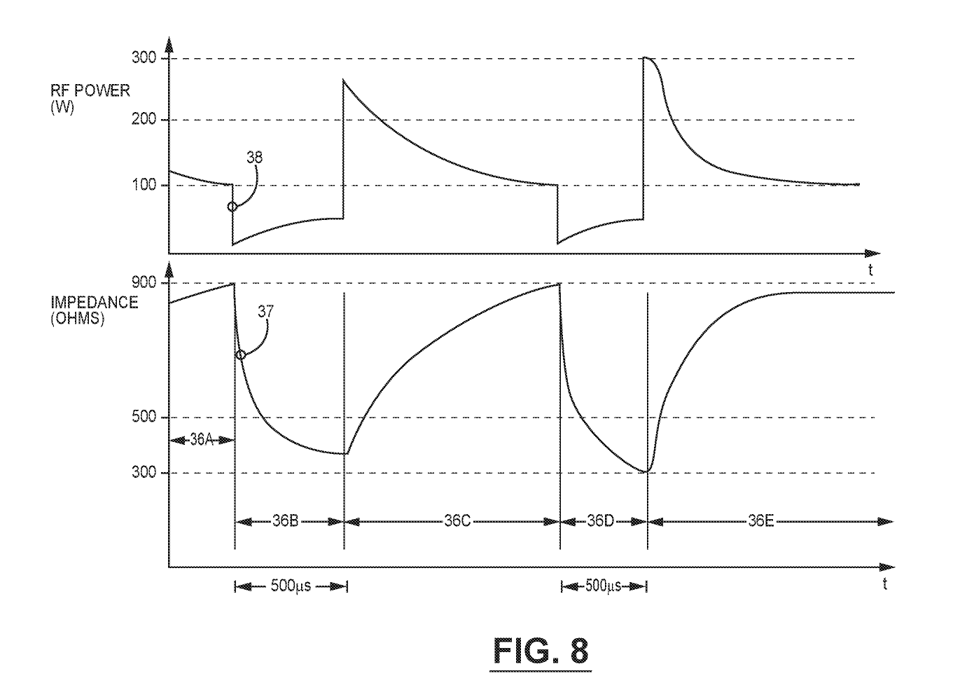

FIG. 8 includes time traces for RF power 38 delivered to the RF electrode tip and the impedance 37 between poles of the RF electrode during operation of a hemostatic waveform algorithm. The different time intervals of the hemostatic waveform are delineated by 36A through 36E. During interval 36A there is an active RF cutting plasma present, and due to the impedance 37 between electrode poles rising to a threshold of 900 Ohms the power 38 delivered to the electrode at 300 Vrms is observed to fall towards 100 W. This condition is likely to arise when the loop of the RF electrode has an excessively deep engagement with prostate gland tissue, with increased risk of more rapid transection of a number of blood vessels. To slow down the cutting process and increase the thermal margin associated with cutting, as the RF impedance 37 between electrode poles exceeds 900 Ohms, the RF output voltage is dropped from 300 Vrms to 120 Vrms. At the start of the next time interval 36B, the plasma is immediately extinguished due to insufficient voltage amplitude, and the impedance 37 between RF electrode poles is seen to rapidly fall as the vapor gap around the electrode loop collapses. In this instance the impedance 37 between poles of the RF electrode does not fall as low as 300 Ohms, due to the extent of envelopment of the loop electrode within tissue. At the start of interval 36B the power 38 dissipated is immediately dropped to 16% of the power 38 dissipation at the end of the cutting interval 36A. This is the impact of stepping the RF voltage down from 300 Vrms to 120 Vrms. Over the interval 36B this power 38 dissipation rises substantially in inverse linear proportion to the reduction of the impedance 37 between the RF electrode poles. In the instance depicted by interval 36B, a maximum quench duration limit of 500 us is reached before the impedance 37 reaches a lower limit and so the next cut interval 36C is commenced. Due to the extent of RF electrode loop engagement with tissue and the existing temperature elevation around the RF electrode loop, re-establishment of the cutting plasma is much quicker than depicted in the timeline of FIG. 4. For the same reasons the power 38 surge needed to re-establish a plasma is limited typically to less than 300 W. This RF power 38 dissipation rapidly decays again towards 100 W as the vapor gap is re-established around the RF electrode loop, immediately followed by a cutting plasma. Interval 36D is similar to interval 36B, with the distinction that this depicts a lesser engagement between tissue and the RF electrode loop. This is also seen when higher precision is required, such as bladder procedures. The corollary of this reduced contact with tissue is that there is a greater proportion of the RF electrode loop immersed in saline as soon as the vapor gap has collapsed. As a consequence, the impedance 37 between RF electrode poles is more a function of extent of collapse of the vapor gap around the RF electrode loop. Interval 36D is therefore terminated on the basis of the impedance falling below 300 Ohms, before the 500 us timeout is reached. In interval 36E, the greater exposure of the RF electrode loop to saline results in a greater surge of power 38 being required to re-establish a plasma, and indeed the impedance between electrodes is depicted as then not reaching 900 Ohms and as a consequence the RF output remains in the cutting state at 300 Vrms. Note that for procedures with more superficial engagement between the loop and tissue such as for bladder tumours, the upper impedance limit allowable for the cutting intervals may be dropped significantly, for example to as low as 600 Ohms, with the effect of making hemostatic waveform pulsing the more likely.

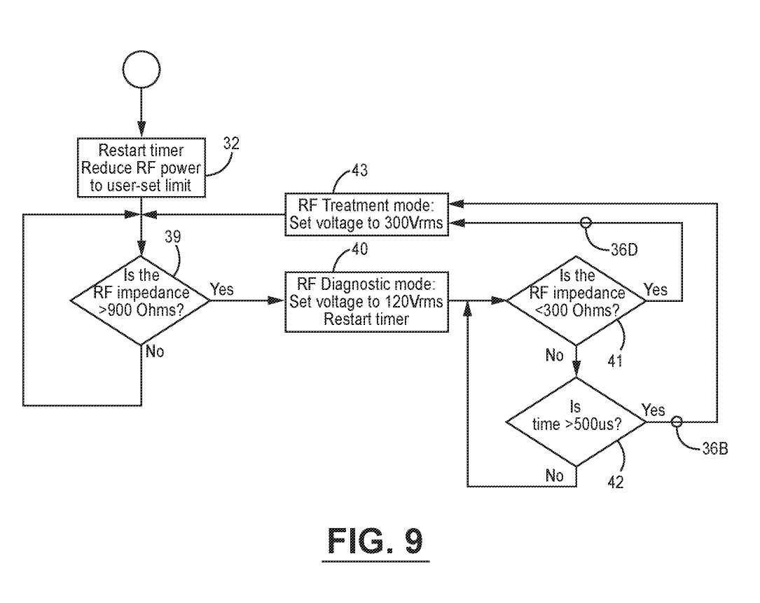

FIG. 9 includes an algorithm to implement the control of cut and quench states depicted in FIG. 8 by time traces for power and impedance. The algorithm starts at step 32, the end of the power surge interval in FIG. 5. For clarity, per step 32 in FIG. 5, at step 32 in FIG. 9 the RF voltage has been set to 300 Vrms for cutting. The impedance between RF electrode poles is repeatedly checked for exceeding 900 Ohms at step 39. If the impedance does exceed 900 Ohms while in the cut state, in FIG. 9 also referred to as the RF treatment mode, the RF voltage is set to 120 Vrms at step 40 and the quenched state or diagnostic mode is entered. There are 2 routes to exit this state. The first is at step 41 if the impedance falls below 300 Ohms, but failing that at step 42, after a 500 us timeout the RF treatment mode is restarted at step 43. The two routes back from the diagnostic mode to the RF treatment mode are labelled as 36B and 36D and correspond to the decision pathways at the ends of said intervals in FIG. 8.

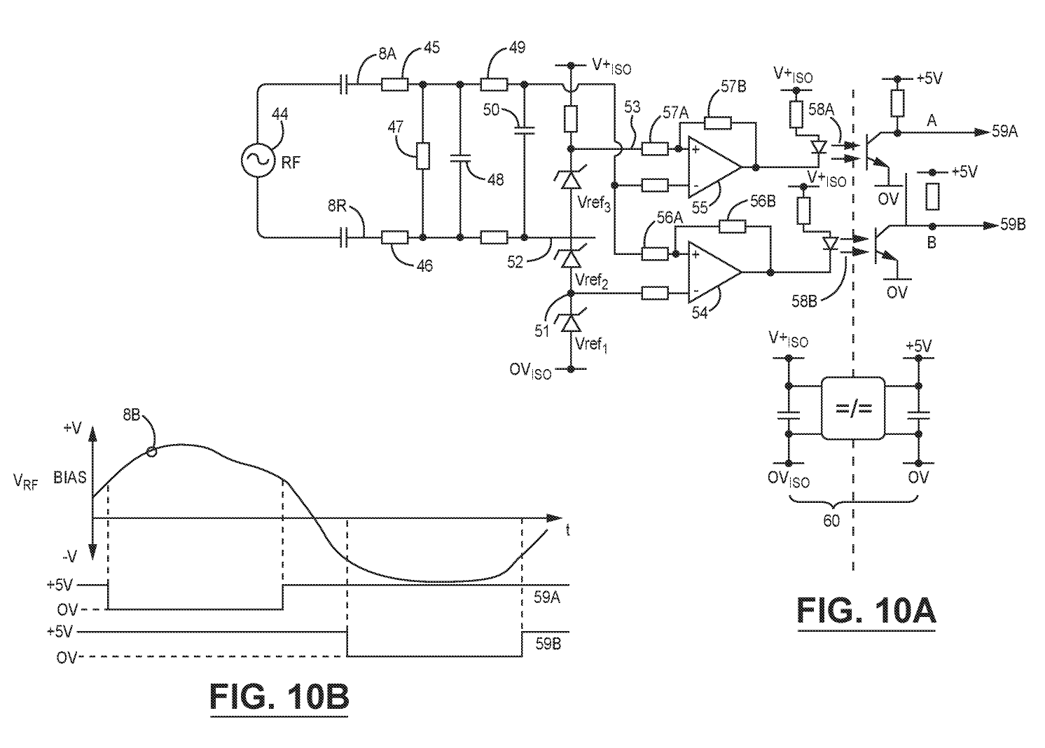

The circuit in FIG. 10A is designed to detect the polarity of the dc bias at the RF output when it exceeds a preselected threshold. The electrosurgical generator RF source is generically depicted by 44. This is capacitively coupled to the RF output active 8A and return 8R poles with connection to the RF electrode via an interconnecting cable as shown in FIG. 1. There is a floating reference circuit connected across the RF output poles 8A and 8R and these necessarily have a very high input impedance 45,46 of several Mega-Ohms, to ensure that is not possible to draw more than 10 uA of dc current under the effect of the DC bias voltage caused by partial rectification of the RF ac waveform applied to the RF electrode. The network of capacitors and resistors included in 45 to 50 serve to divide down the dc voltage and to filter out the RF component present between the RF output poles 8A, 8R. Circuit nodes 51 and 53 are at thresholds respectively above and below node 52 and as such comparators 55 and 54 will respectively drive opto coupler LEDs 58A or 58B if the dc bias at the RF output poles 8A,8R is more positive or more negative than the threshold limits defined at nodes 53 and 51. When either optocoupler LEDs 58A or 58B are driven, the signals 59A or 59B go from a logic-high state to a logic-low state, corresponding to a positive or a negative dc bias between the RF output poles 8A, 8R. For each of the comparators 55 or 54 there is a hysteresis resistor network 57A, 57B or 56A, 56B which prevents slowly changing dc bias levels from causing noisy transitions at 59A or 59B.

The time traces in FIG. 10B show the response of the circuit at 59A and 59B to first positive and then negative dc biases between the RF output poles. Using the signals 59A and 59B, the microcontroller algorithms can deduce if there is an appreciable plasma present and deduce if it swaps between the RF electrode active pole and return pole.

While the invention has been particularly shown and described as referenced to the embodiments thereof, those skilled in the art will understand that the foregoing and other changes in form and detail may be made therein without departing from the spirit and scope of the invention.

What is claimed is:

1. An electrosurgical generator for supplying radio frequency (RF) power to an electrosurgical instrument that has been introduced to the surgical site for cutting or vaporizing tissue immersed in a saline medium, the generator comprising:
an internal RF delivery stage able to deliver more than 55 Joules of energy to the electrosurgical instrument within 110 ms; and an internal storage capacity associated with RF waveform supply of less 5 Joules.

2. A generator according to claim 1 where the RF stage is able to deliver up to 110 Joules of energy within 145 ms

3. A generator according to claim 1 where the RF stage is able to deliver up to 110 J of energy within 110 ms.

4. A generator according to claim 3 where the RF stage is able to deliver up to 230 J of energy within 320 ms.

5. A generator according to any preceding claim incorporating an RF waveform synthesis stage including at least 1 pair of RF switching transistors.

6. A generator according to any preceding claim incorporating an RF synthesis stage including at least 2 pairs of RF switching transistors.

7. A generator according to claim 6 where the transistors are configured as an H bridge circuit comprised of 2 half bridge pairs of transistors.

8. A generator according to any preceding claim with a maximum peak RF voltage of less than 500V and a maximum root mean square voltage of less than 360 Vrms.

9. A generator according to any preceding claim, with a maximum output current in excess of 3A root mean square with a RF current measurement sensor coupled to a control circuit able to disable the unipolar (dc) supply to the RF stage within ½ of the RF cycle upon detection of an electrosurgical instrument current in excess of an allowable limit.

10. A generator according to any of claims 1-8, with an RF current measurement sensor coupled to a control circuit able to within ½ of the RF cycle, alter the switching pattern of the RF transistors such that the voltage difference between the centre point nodes of the 2 half bridge pairs remains substantially zero, but the impedance between centre point nodes via 2 of the 4 switching transistors remains less than 1 Ohm.

11. A generator according to any preceding claim incorporating a means of computing the energy delivered to the electrode over a time interval, where the energy delivery rate is dropped to less than 300 W outside the specified power surge intervals of claims 1-4.

12. A generator according to claims 1-11 incorporating a means of computing the energy delivered to the electrode over a time interval, where the energy delivery rate is dropped to less than 160 W outside the specified power surge intervals of claims 1-4.

13. A generator according to any preceding claim with a time constrained power surge interval incorporating a means of computing the impedance between the electrode poles, where RF delivery is stopped upon detection of an unacceptable impedance indicative of an absent or incomplete vapor gap or plasma within the power surge interval or optionally an impedance settling delay thereafter; with an impedance settling delay of up to 1 second; and with an unacceptable impedance being one of less than 300 Ohms and preferably less than 600 Ohms.

14. A generator according to any preceding claim, including a means of computing the impedance between the poles of the electrode where upon initial activation of RF delivery, a during a diagnostic interval preceding commencement of RF treatment; an RF voltage of less than 180 Vrms is applied, with RF treatment commencing only if the measured impedance falls within an acceptable range.

15. A generator according to claim 14 where the minimum acceptable impedance during the diagnostic time interval has a value between 10 and 180 Ohms.

16. A generator according to claim 14 where the minimum acceptable impedance during the diagnostic time interval has a value between 20 and 180 Ohms.

17. A generator according to claim 14 where the minimum acceptable impedance during the diagnostic time interval has a value between 100 and 180 Ohms.

18. A generator according to any preceding claim where the maximum acceptable impedance during the diagnostic time interval has a value between 20 and 400 Ohms.

19. A generator according to any preceding claim where the maximum acceptable impedance during the diagnostic time interval has a value between 20 and 60 Ohms.

20. A generator according to any preceding claim with the generator alternating between a first RF plasma delivery mode and a second RF non-plasma delivery mode;
with the waveform voltage amplitude during the RF plasma delivery mode being greater than 220 Vrms; and
the voltage during RF non-plasma delivery mode being less than 180 Vrms;
wherein the generator remains in RF plasma delivery mode until an RF plasma mode impedance limit is measured to have been exceeded whereupon it switches to the RF non-plasma mode; and
the generator remains in RF non-plasma mode until the impedance falls below a RF non-plasma mode impedance limit (indicative of plasma vapor gap collapse), or until a maximum non-plasma mode interval has elapsed.

21. A generator according to claim 20 where the RF plasma mode impedance limit is greater than 750 Ohms.

22. A generator according to claim 20 where the RF plasma mode impedance limit is greater than 900 Ohms.

23. A generator according to claim 20 where the RF plasma mode impedance limit is adjustable between 400 and 1600 Ohms by a sensitivity user adjustment.

24. A generator according to any of claims 20-23 where the RF non-plasma mode impedance limit is less than 400 Ohms.

25. A generator according to any of claims 20-23 where the RF non-plasma mode impedance limit is less than 120 Ohms.

26. A generator according to any of claims 20-23 where the RF non-plasma mode impedance limit is adjustable between 40 and 600 Ohms via a sensitivity user adjustment.

27. A generator according to any of claims 20-26 where the maximum non-plasma mode interval is between 250 us and 4 ms.

28. An electrosurgical generator for supplying radio frequency (RF) power to an electrosurgical instrument that has been introduced to the surgical site for cutting or vaporizing tissue immersed in a saline medium
with a maximum peak RF voltage of less than 500V and a maximum root mean square voltage of less than 360 Vrms
wherein the electrosurgical generator RF transistors are configured as an H bridge circuit comprised of 2 half bridge pairs of transistors
with a maximum output current in excess of 3A root mean square
with an RF current measurement sensor coupled to a control circuit able to within ½ of the RF cycle, alter the switching pattern of the RF transistors such that the voltage difference between the centre point nodes of the 2 half bridge pairs remains substantially zero, but the impedance between centre point nodes via 2 of the 4 switching transistors remains less than 1 Ohm.

29. An electrosurgical generator for supplying radio frequency (RF) power to an electrosurgical instrument that has been introduced to the surgical site for cutting or vaporizing tissue, the electrosurgical instrument comprising at least 2 poles, the RF output of the generator being coupled to the electrosurgical instrument by at least 2 conductors, the generator comprising:
a series coupling capacitance between the RF source and the connections to the electrosurgical instrument
a means of measurement of the polarity of dc bias appearing between the poles of the electrosurgical instrument
a means of disabling the RF output in response to one or more adverse polarities between the poles of the electrosurgical instrument.

30. An electrosurgical generator for supplying radio frequency (RF) power to an electrosurgical instrument that has been introduced to the surgical site for cutting or vaporizing tissue, the electrosurgical instrument comprising at least 2 poles, the RF output of the generator being coupled to the electrosurgical instrument by at least 2 conductors, the generator comprising:
a series coupling capacitance between the RF source and the connections to the electrosurgical instrument
a means of measurement of the polarity of dc bias appearing between the poles of the electrosurgical instrument
a means of annunciating an alarm in response to one or more adverse polarities between the poles of the electrosurgical instrument.

31. An electrosurgical system according to 29 or 30, where the adverse polarity is defined a positive voltage at the active pole or poles relative to the return pole or poles of the electrosurgical instrument.

32. An electrosurgical system according to 29 or 30, where the adverse polarity is defined a negative voltage at the active pole or poles relative to the return pole or poles of the electrosurgical instrument.

33. An electrosurgical system according to 31 or 32, where the adverse polarity is defined a change in polarity during RF activation of the voltage at the active pole or poles relative to the return pole or poles of the electrosurgical instrument.

34. An electrosurgical generator for supplying radio frequency (RF) power to an electrosurgical instrument that has been introduced to the surgical site for cutting or vaporizing tissue immersed in a saline medium
with a normal cutting or vaporizing interval with maximum peak RF voltage of less than 500V and a maximum root mean square voltage of less than 360 Vrms
with a preamble interval following initial RF activation and preceding normal cutting or vaporization with a diagnostic voltage of less than 180 Vrms during the preamble interval
wherein impedances measured during the preamble interval should be both greater than a lower limit and less than an upper limit to allow commencement of the normal cutting or vaporizing interval.

35. An electrosurgical system according to 34 where the lower limit is not greater than 20 Ohms.

36. An electrosurgical system according to 34 where the upper limit is not less than 290 Ohms.

37. An electrosurgical system according to 35 and 36.
